(19) United States
(12) Patent Application Publication
(10) Pub. No.: US 2006/0074295 A1
(43) Pub. Date: Apr. 6, 2006

(54) Combined MR coil technology in medical devices

(75) Inventors: John Kucharczyk, Bishop, GA (US); Michael John Bronskill, Toronto (CA)

Correspondence Address: Mark A. Litman & Associates, P.A.;Suite 205, 3209 West 76th St., York Business Center, Edina, MN, 55435, US

(73) Assignees: NexGen; Sunnybrook & Women's College Health Sciences Centre

(21) Appl. No.: 10/957,538

(22) Filed: Oct. 1, 2004

(51) Int. Cl. A61B 5/05

(52) U.S. Cl. 600/422

(57) ABSTRACT

A method, system and apparatus provides a magnetic resonance (MR) responsive field of view within a volume of a patient. At least two radiofrequency (RF) surface coils are provided that at least in part MR responsively cover the volume, at least one MR responsive microcoil is provided within the volume, and MR responsive fields are simultaneously generated from the at least two RF surface coils and the at least one microcoil. The data of the RF responsive fields are then integrated with parallel imaging methods.

### BACKGROUND OF THE INVENTION

1. Field of the Invention

The present invention relates to the field of medical devices, medical devices that are used within organisms, medical devices that are used with or incidental to Magnetic Resonance Imaging, or medical devices that are used with medical treatments after, during or preceding Magnetic Resonance Imaging of the region within which the treatment is planned.

2. Background of the Art

Medical procedures may now be performed on areas of the patient which are relatively small. Procedures may be performed on small clusters of cells, within veins and arteries, and in remote sections of the body with minimally invasive techniques, such as without surgical opening of the body. As these procedures, such as balloon angioplasty, microsurgery, electrotherapy, and drug delivery are performed within the patient with minimally invasive techniques without major surgical opening of the patient, techniques have had to be developed which enable viewing of the procedure concurrent with the procedure. X-ray imaging, such as X-ray fluoroscopy, is a possible method of providing a view of the procedural area, but X-ray exposure for any extended period of time is itself harmful to the patient. Fiber optic viewing of the area does not provide any harmful radiation to the patient, but the fiber optics may take up too large a space to provide both the light necessary for viewing and a path for return of the light, and does not permit imaging beyond the surface (that is, only the surfaces of internal objects may be viewed from the position where the fiber optic device is located). Fiber optic viewing or direct light viewing is more acceptable for larger area medical procedures such as gastroenterological procedures than for more microscopic procedures such as intraparenchymal drug delivery or endovascular drug delivery or procedures. Techniques have been developed for relatively larger area viewing of MR-compatible devices within a patient by the use of MR-receiver coils in the devices which are tracked by MR imaging systems. Few specific design considerations have been given to devices which have MR viewing capability and specific treatment functions, especially where the relationship of specific types of treatment and the MR receiver coils must be optimized both for a treatment process and for MR viewing ability.

U.S. Pat. No. 5,211,165 describes a tracking system to follow the position and orientation of an invasive device, and especially a medical device such as a catheter, using radio frequency field gradients. Detection of radio frequency signals is accomplished with coils having sensitivity profiles which vary approximately linearly with position. The invasive device has a transmit coil attached near its end and is driven by a low power RF source to produce a dipole electromagnetic field that can be detected by an array of receive coils distributed around an area of interest of the subject. This system places the transmit coils within the subject and surrounds the subject with receive coils.

U.S. Pat. No. 5,271,400 describes a tracking system to monitor the position and orientation of an invasive device within a subject. The device has an MR active sample and a receiver coil which is sensitive to magnetic resonance signals generated by the MR active sample. These signals are detected in the presence of magnetic field gradients and thus have frequencies which are substantially proportional to the location of the coil along the direction of the applied gradient. Signals are detected responsive to sequentially applied mutually orthogonal magnetic gradients to determine the device's position in several dimensions. The invasive devices shown in FIGS. 2a and 2b and RF coil are an MR active sample incorporated into a medical device and an MR active sample incorporated into a medical device, respectively. U.S. Pat. No. 5,375,596 describes a method and apparatus for determining the position of devices such as catheters, tubes, placement guidewires and implantable ports within biological tissue. The devices may contain a transmitter/detector unit having an alternating current radio-frequency transmitter with antenna and a radio signal transmitter situated long the full length of the device. The antennae are connected by a removable clip to a wide band radio frequency (RF) detection circuit, situated within the transmitter/detector unit.

U.S. Pat. No. 4,572,198 describes a catheter for use with MR imaging systems, the catheter including a coil winding for exciting a weak magnetic field at the tip of the catheter. A loop connecting two conductors supports a dipole magnetic field which locally distorts the NMR image, providing an image cursor on the magnetic resonance imaging display.

U.S. Pat. No. 4,767,973 describes systems and methods for sensing and movement of an object in multiple degrees of freedom. The sensor system comprises at least one field-effect transistor having a geometric configuration selected to provide desired sensitivity.

U.S. Pat. Nos. 5,451,774 and 5,270,485 describe a three-dimensional circuit structure including a plurality of elongate substrates positioned in parallel and in contact with each other. Electrical components are formed on the surfaces of the substrates, along with electrical conductors coupled to those components. The conductors are selectively positioned on each substrate so as to contact conductors on adjacent substrates. The conductor patterns on the substrates may be helical, circumferential, or longitudinal. Radio frequency signaling between substrates would be effected with a transmitting antenna and a receiving antenna, with radio frequency signal transmitting and receiving circuitry present in the substrates (e.g., column 7, lines 32-43). Circulation of cooling fluid within the device is shown.

U.S. Pat. No. 6,023,166 (Eydelman) describes a radio frequency antenna for conducting magnetic resonance imaging studies of the breast region of a patient which includes a tuned primary coil inductively coupled to two tuned secondary coils. Each secondary coil defines a region for receiving one of the breasts of the patient, and receives magnetic resonance signals emitted from each breast and the surrounding region of the patient. The primary coil can be connected to the receiving circuitry of the magnetic resonance imaging system. The magnetic resonance signals received by the secondary coils induce signals in the primary coil which are provided to the magnetic resonance imaging system for processing. The secondary coils, which preferably include two windings, each have a portion adjacent to the primary coil and a portion distanced from the primary coil. The portions of the secondary coils adjacent to the primary coils preferably lie in substantially the same plane as the primary coil, while the portion of the secondary coil distanced from the primary coil lies in a second plane intersecting the plane of the primary coil. A cushion arrangement for supporting an antenna, and a method for collecting magnetic resonance signals from the breast region of a patient, are also disclosed.

Published U.S. Patent Application 20030132750 describes a magnetic resonance imaging system which includes an MR signal reception apparatus comprising a receiving multi-coil and a switchover member. The receiving multi-coil receives MR signals and is composed of a plurality of element coils. The switchover member is configured to switch reception states of the MR signals received by the plurality of element coils in response to imaging conditions. The switchover member connects output paths of the MR signals from the plurality of element coils to reception channels in the receiver in response to the imaging conditions. The reception channels are less in number than the element coils. The imaging conditions are for example directed to parallel MR imaging. The use of parallel imaging is also discussed with the coils.

Published U.S. Patent Application 20030030437 describes a magnetic resonance imaging apparatus in which k-space data received from RF excitation pulses applied at successive phase-encode gradients and read-out while other gradients are applied is collected for individual coils of an array of RF receive coils. A processor uses the lines of data received by each RF receive coil at each phase-encode gradient together with reference spatial sensitivity profiles of each coil in a phase-encode direction represented in terms of spatial harmonics of a fundamental frequency one cycle of which corresponds with a desired field of view, to generate a set of phase-encode lines. These lines are converted to image space in Fourier Transform processor to produce an image for display on monitor. Smash parallel imaging technology is used with the coils.

Published U.S. Patent Application 20030094948 describes parallel imaging with multiple surface coils with three-dimensional arrangement of the surface coils.

Published U.S. Patent Application 20020158632 describes that advanced processing techniques can be used to enhance the robustness, efficiency, and quality of several parallel imaging techniques, such as SMASH, SENSE and sub-encoding. Specifically, a magnetic resonance image is formed by measuring RF signals in an array of RF coils, forming a set of spatial harmonics and tailoring the set of spatial harmonics to form a set of tailored spatial harmonics that are adjusted for variations in at least one of angulation of an image plane, field of view, and coil sensitivity calibration. The harmonics may be tailored by selecting automatically a subset of the set of formed spatial harmonics, adjusting the set of spatial harmonics by a function not equal to 1, to adjust for sensitivity variations along a phase encode direction, and/or performing separate spatial harmonic fits of the coil sensitivities at different spatial positions to the set of tailored spatial harmonics. The magnetic resonance image may also be formed by generating a set of encoding functions representative of a spatial distribution of receiver coil sensitivities and spatial modulations corresponding to the gradient encoding steps, transforming the set of encoding functions to generate a new set of functions representative of distinct spatial positions in the image, and applying the new set of functions to a set of MR signals to form the magnetic resonance image. Matrices inverted during the process of forming the magnetic resonance image may be conditioned by thresholding the eigenvalues of the matrix prior to inversion.

### SUMMARY OF THE INVENTION

A method, system and apparatus provides a field of view within a volume of a patient. At least two RF surface coils are provided that at least in part MR responsively cover the volume, at least one MR responsive microcoil is provided within the volume, and MR responsive fields are simultaneously generated from the at least two RF surface coils and the at least one microcoil. The data of the RF responsive fields are then integrated with parallel imaging methods.

Magnetic Resonance (MR) microcoil imaging methods can be used to assist in the determination of devices, materials and/or tissues located or delivered around devices within an organism. Some advanced techniques described above localize delivery of therapeutic agents, including drugs, stem cells, and gene vectors, and can assist in estimating the migration and spatial temporal distribution of the therapeutic agents within tissues. Active MR visualization of drug and cell delivery can be achieved by means of one or more radio frequency (RF) microcoils positioned in a medical device inserted into an organism. One location may be, by way of non-limiting examples, along the longitudinal axis of the delivery device. Single microcoils may be used separately (with another microcoil associated with the medical device not being active or being sensitometrically ignored) or the combination of microcoils may constructed in an array that may be used together to optimally image the surrounding environment, including the tissue structure and function within the field of response of the microcoils. The system of coils with the devices may, by way of non-limiting example, be used for very small (picoliter, nanoliter or microliter) injections measured within a solenoid volume RF microcoil, which by design is mainly sensitive to the volume inside the coil. The imaging volume in such a use is usually directly related to the diameter of the RF coil.

A combination of RF microcoil(s) and surface coil(s) can be used with a preamplifier positioned on the delivery device (or distally connected by electronics or wires/cables to the combination of microcoils), which serves to amplify signals from the RF microcoils. More than one surface coil and more than one microcoil may be present, as the distribution of microcoils along a length of the image area or the medical device (e.g., a catheter) helps to define the adjacent region(s) better within which local signals are detected. The coils may add or integrate or otherwise combine their detectable volumes, thereby defining a combined volume (the term “combined volume” will be used to mean the field volumes produced by both the surface coil(s) and the microcoil(s) used during imaging) which can be efficiently observed by the MR system.

Parallel imaging methods are defined as various MRI techniques, outlined above, for example, and originally designed to reduce the scan time. The reduction is achieved by under-sampling k-space and recording images simultaneously from multiple imaging coils. Using information about the sensitivity patterns of the multiple imaging coils, it is possible to solve a set of simultaneous equations to piece together an image of the fields of view of the coils. In general, but not exclusively, parallel imaging methods thus measure the sensitivity patterns of coils, fill/build k-space more intelligently, and provide artifact reduction and non-uniformity correction in processing simultaneous signals to produce MR images. The use of parallel imaging methods with the combined volume produces an enhanced field volume for view that includes high signal-to-noise ratio data and includes multiple field regions (e.g., adjacent and forward from the front end of a medical device and/or surrounding the device along its length to cover a region in which the medical procedure is to be performed). As different medical procedures are performed in different environments, the coils may be located, sized, angled, distributed or otherwise designed to provide specific MR signals, fields and/or responses tailored to the anticipated needs of a particular procedure. In general, the technology described herein may be practiced by employing an array of RF microcoils in combination with an array of surface coils, such that images are obtained at many orientations to the delivery device.

Magnetic resonance is a low sensitivity technique; consequently the signal-to-noise (SNR) ratio available for a particular set of experimental or working conditions determines the minimum spatial resolution achievable. In one of the various methods that may be practiced within the generic scope of the present invention, for real-time monitoring of stem cell or drug delivery into the brain, MR images are acquired with an in-plane resolution of no more than 1 mm and a temporal resolution of, by way of non-limiting examples, less than 100 ms, such as within the ranges of either 10-80 ms, 20-70 ms, or 30-40 ms. These imaging requirements are addressed in practice through progressive implementation of accelerated imaging acquisition methods combined with spatial and temporal targeted data. Parallel imaging methods, which take advantage of the varied spatial sensitivity of multiple receiver channels to reduce the total data required, can be used in the method of the invention. Real-time SENSE imaging using a hardware optimized echo-planar pulse sequence with an 8-channel data acquisition system may be used to image at, by way of a non-limiting example, 52 ms per frame for a 256×120 matrix over a 36×28 field-of-view.

A number of independent and severable perspectives or aspects of the technology described herein may be viewed from a non-limiting consideration. One aspect of this technology enables the use of parallel, simultaneous or coincident MR imaging methods on the combination of surface coil(s) and microcoil(s) that provide for high-resolution imaging adjacent to (within the range of generated fields or within a useful distance of the generated fields where information from the fields may be useful) the microcoil(s) and the field of treatment or view to enable accurate characterization by MR methods of the initial location for treatment, diagnosis or therapeutic agent delivery.

A second aspect of the present technology enables parallel imaging methods and the combination of surface coil(s) and microcoil(s) to extend the field-of-view beyond the imaging volume of the microcoil(s) to enable dynamic MR visualization of the migration of therapeutic agents over hours and days after delivery.

A third aspect of this technology enables integrated MR imaging/MR spectroscopy that enables quantitative mapping of the spatial distribution kinetics of injected therapeutic agents in relation to site-specific changes in tissue biochemistry.

A fourth aspect of this technology enables enhanced MR imaging by adding or integrating the imaging field-of-view of RF microcoils and surface coils, thereby defining a combined volume which can track device location and subsequently the spatial distribution of injected therapeutic agents periodically or over hours and days after tissue delivery.

A fifth aspect of this technology enables enhanced MR imaging by employing an array of RF microcoils in combination with an array of surface coils, such that images are obtained for any orientations of the delivery device(s).

A sixth aspect of the present technology enables improved spatial and temporal MR imaging of drug delivery and distribution through progressive implementation of parallel imaging methods, wherein hardware optimized trapezoidal gradient pulses allow a reduction in scan time of, by way of non-limiting examples, 20%, 30%, 40% or more over typical segmented k-space methods with limited reduction of SNR.

A seventh aspect of the technology described herein enables the use of parallel imaging methods which take advantage of the varied spatial sensitivity of multiple receiver channels to reduce the total data required to produce images from both microcoils and surface coils.

An eighth aspect of the present technology is to use MR microcoils and MR surface coils interactively and simultaneously, to eliminate the current “searchlight in the dark” problem of imaging with only a single microcoil.

A ninth aspect of this technology is to use parallel MR imaging methods to preferentially enhance spatially varying SNR in the region of the microcoil(s).

A tenth aspect of the technology is to use surface coils instead of the head coil for higher SNR over a smaller region, with better access simultaneously to the patient's cranial region for interventional purposes.

### BRIEF DESCRIPTION OF THE FIGURES

FIG. 1 is a diagram of a drug, cell or gene therapy infusion catheter incorporating a microcoil connected to an MR imager.

FIG. 2 illustrates one embodiment of the present invention comprising an infusion catheter with a microcoil which has an RF responsive field radially disposed around the microcoil position adjacent to the distal end of the catheter.

FIG. 3 illustrates another embodiment of the invention comprising an infusion catheter that incorporates two microcoils each having an RF responsive field radially disposed around the microcoil position.

FIG. 4 illustrates a further embodiment of the invention comprising a microcoil within a catheter device that is surgically inserted into the brain of a human patient using MR imaging guidance. The catheter and microcoil are surrounded by two surface coils. The MR responsive signals from each coil interface with separate receiver channels of an MR imager.

FIG. 5 illustrates another embodiment of the invention comprising an infusion catheter with an RF microcoil and two RF surface coils. Signals from the surface coils and microcoil interface with separate receiver channels of an MR imager.

FIG. 6 illustrates a preferred embodiment of the method of the invention comprising an infusion catheter with microcoil surgically inserted into a human brain with two surface coils positioned around the insertion point of the catheter. According to the invention, signals from the surface coils and microcoil input receiver channels of an MR imager wherein parallel imaging methods are used to extend the field-of-view beyond the imaging volume of the microcoil to enable dynamic MR visualization of the migration of therapeutic agents delivered from the infusion catheter over hours and days after delivery.

FIG. 7 illustrates an infusion catheter and microcoil with signal leads extending to an MR imager. According to the invention, an RF responsive field radially disposed around the microcoil position covers the volume of infusion during the immediate post-infusion period.

FIG. 8 illustrates an infusion catheter with microcoil, wherein the RF field radially disposed around the microcoil does not cover the entire volume of a drug infusion when the volume of the infusions is large.

FIG. 9 illustrates a further preferred embodiment of the invention wherein MR responsive signals from a catheter-based microcoil are integrated with MR responsive signals from surface coils positioned on the head using parallel imaging methods. According to the invention, the combined imaging volume of the microcoil and surface coils enables tracking of device location and subsequently the spatial distribution of injected therapeutic agents periodically or over hours and days following catheter delivery.

FIG. 10 illustrates another preferred embodiment of the invention wherein parallel imaging methods are used to produce a combined MR imaging volume from the three surface coils and the internal microcoil.

### DETAILED DESCRIPTION OF THE INVENTION

A technology is practiced including a medical device having a longitudinal axis, the device having at least one RF microcoil system in the device, at least one surface coil system around the regions (e.g., patient, organ, member, etc.) to be imaged, and a parallel imaging method support system (e.g., sufficiently advanced MR system as is known in the art for use in parallel imaging,, software presently available with commercial imaging systems for use in parallel imaging methods, and a viewing system (e.g., CRTY, plasma screen, hard copy media, LED display, etc.). The position of the at least one RF microcoil system and the at least one surface coil system with respect to the longitudinal axis and the patient define regions where different field volumes are provided by the at least one RF microcoil system and the at least one surface coil system. The medical device may have the surface microcoil system connected with at least one preamplifier in communication with a signal receiving system and the at least one surface coil system may have at least one preamplifier in communication with a signal receiving system. The medical device may have at least one microcoil system and at least one surface coil system, and/or there are at least two distinct RF microcoil systems that provide image data that can be treated by parallel processing. In parallel imaging methods, a reduced data set in the phase encoding direction(s) of k-space is acquired to shorten acquisition time. The spatial information related to the at least one surface coil system and the at least one microcoil system are utilized for reducing conventional Fourier encoding.

First, low-resolution, fully Fourier-encoded reference images are required for sensitivity assessment. Parallel imaging reconstruction in the Cartesian case is efficiently performed by creating one aliased image for each array element using discrete Fourier transformation. The next step then is to create a full-FOV image from the set of intermediate images. In principle, parallel imaging methods can be applied to any imaging sequence and k-spacetrajectory. These techniques are variously named in the art as, e.g., SENSE, IPAT, SMASH, SPEEDER, ASSET.

Parallel imaging methods can also make use of arrays of coils. The phased array coils are typically operated as “receive only” coils. A larger coil on the imager is used as the transmitter of RF energy to produce the 90° and 180° pulses. State-of-the-art coil systems include the use of eight or more coils with eight separate receivers. This method is often referred to as a phased array system, although the signals are not added such that the signal phase information is directly included. The use of phased array coils allows the number of signal averages to be decreased with their superior SNR and resolution, thereby decreasing scan time. Fast parallel imaging methods using surface phased array multichannel coils, and image acquisition and reconstruction schemes such as sensitivity encoding (SENSE) or simultaneous acquisition of spatial harmonics (SMASH) can further improve spatial and temporal resolution. Parallel imaging methods make use of spatial sensitivity differences between individual coils in an array to reduce the gradient encoding required during image acquisition. This reduces acquisition times by decreasing the number of phase-encoded lines of k-space that must be acquired. There are several distinct classes of practical implementation of parallel imaging, three of which are known as SENSE (Magnetic Resonance in Medicine 42: 952-962 (1999)—SENSE: Sensitivity Encoding for Fast MRI by Klaas P. Pruessmann, Markus Weiger, Markus B. Scheidegger and Peter Boesiger), SMASH (WO-A-98/21600 and Magnetic Resonance in Medicine 38: 591-603 (1997)—Simultaneous Acquisition of Spatial Harmonics (SMASH): Fast Imaging with Radiofrequency Coil Arrays by Daniel K Sodickson and Warren J Manning) and SPACE-RIP (WO-A-00/72050 and Magnetic Resonance in Medicine 44: 301-308 (2000)—Sensitivity Profiles from an Array of Coils for Encoding and Reconstruction in Parallel (SPACE RIP) by Walid E Kyriakos, Laurence P Panyah, Daniel F Kaches, Carl-Frederick Westin, Sumi M Bao, Robert V Mulkem and Ferenc A Jolesz). All of these methods require information about the coil sensitivity profiles (reference data), which is used to regenerate a full image data set from the sub-sampled k-space acquisition (target data). These references are incorporated herein by reference in their entirety for their technical disclosure relating to parallel imaging methods and the apparatus, software and hardware that can be used to effect parallel imaging methods.

SENSE is a form of parallel imaging method that operates in the image domain for both the target image data and the coil reference data. The method can be used with a wide range of coil geometries. A typical receive coil arrangement comprises coils placed on opposite sides of the patient or surrounding the patient. Multiple surface coils are desirable in parallel imaging methods so that the coils have different fields of view. The target data is acquired for each receive coil with a reduced field of view, which results in aliasing, so that each coil produces a k-space representation, which can be Fourier Transformed into an aliased image. The aliased images may be then unfolded to the full field of view on a pixel by pixel basis using reference data, which records the relative responses of the receive coils. Reduced field of view imaging imposes a requirement of uniformly spaced samples in the phase-encode direction in k-space. Since processing concerned with unfolding is done in the image domain, individual pixels in the reduced field of view data get unfolded by integer numbers of final pixels (i.e. 1→1, 1→, 1→3 etc). This requires solution of a set of linear simultaneous equations in which pixel intensities are weighted by the coil sensitivity at the final pixel locations. The numerical condition of these equations determines the local noise properties of the unfolded image, so that the signal-to-noise ratio (SNR) varies from pixel to pixel. The signal-to-noise ratio is better in the regions where no aliasing occurs than where it does occur. The resulting patterns of noise variation generally reflect the coil geometry.

SPACE RIP uses k-space target data as input in conjunction with a real space representation of the coil sensitivities to compute directly a final image domain output, that is, the Fourier transform is embedded into the matrix involved. An unfolded image is directly produced from the reduced phase-encode gradient encoded collected data for the coils of the array. Thus, it is a hybrid k-space/real space method and has a higher computational burden than either SENSE or SMASH. It does not require uniform sampling of k-space.

SMASH operates in k-space for the target image data but uses a real space representation of the coil sensitivity profiles. SMASH employs linear combinations of the coil reference data to construct explicitly spatial harmonics that are required to synthesize missing k-space lines. It does not suffer from spatially varying signal-to-noise ratio in the final images, since each point in k-space contributes to the whole image in the image domain.

A typical coil arrangement for SMASH may include an array of coils, e.g., 4-32, 4-24, 4-20, 6-24, 6-20, 8-24, 8-20, 8-20 being ranges of coil numbers that can be easily used and can be compatible with existing software in commercial MRI systems. Among the many available image sections that can be produced (without limiting the types of images can be provided) is to produce a sagittal (vertical longitudinal) section through the spine. The outputs of the individual coils may be suitably weighted and summed. Such a weighted and summed signal modulates received RF signals along the length of the array in the same way as a phase-encoding gradient in the Z-direction modulates RF signals received by an equivalent received coil. Accordingly, SMASH uses weighted combinations of the outputs of the individual coils of the array to simulate the effect of phase-encode gradients on the received RF signals. Different weightings can be used to produce higher harmonics. Thus, signals representing several phase-encode gradient lines can be produced for the application of one phase-encode gradient.

However, SMASH is somewhat restrictive in the coil geometries it can accommodate. In particular, it is not well suited to use with very few coils and the requirement to generate specific spatial harmonics necessitates a given relationship between the imaging field of view and the coil structure. In an arrangement with only two receive surface coils, an anterior coil and a posterior coil arranged above and below a patient in the bore of a magnetic resonance imaging apparatus, there may be an improvement in the image resolution provided from the data of the two surface coils, but it is less improved than the SNR data that would be provided from 6 or more coils.

The technology also may be practiced with a method of providing a magnetic resonance image by:

a) providing a medical device having a longitudinal axis, the device having at least one microcoil internal to the patient and at least one surface coil, the position of the at least one microcoil and the at least one surface coil with respect to the longitudinal axis defining regions where different field volumes are provided by the at least one microcoil and at least one surface coil;

b) placing the device within a magnetic resonance imaging field;

c) generating two distinct imaging fields, at least one field each from the at least one microcoil and at least one surface coil; and

d) integrating images of the two distinct fields into a single image using parallel imaging methods.

Implementation of the combination of the field systems provided by the distinct RF systems and the surface/microcoil systems in each stylistic combination that is designed or provided, would need minimal technology advance from functional support for the individual known systems, and therefore is within the immediate skill and enablement of those in the art when this system has been disclosed and described to them by this specification. The design choices would relate to the terms of number of electronic/communication channels required, the selection of preamp input impedances for individual or combinations of microcoil systems or devices, any extent to which phase encoding steps are reduced, optimization of combined field effects, reduction or optimally designing any overlapping field effects, filtering of any potential cross-talk between signals, artifact reduction techniques, and other minor adjustments within the control of those skilled in the art based on conventional design, software and filtering technology and techniques.

Microcoils tend to generate higher SNR than surface coils (it is believed to be related to the limited field of view and closer coupling to the imaged regions) but over a very small volume or field of view of the region with a very non-uniform sensitivity pattern. With a very small field of view and non-uniform sensitivity, the quality of the image falls off sharply with greater distances from the microcoil(s). The signal from the microcoil rapidly decreases to the level of noise. Microcoil images (on their own) are very small image volumes near the location of the microcoil(s) in the device. The signal pattern from the surface coil(s) and the microcoil(s) according to the technology described herein relates to combination of the at least two fields that are produced (from surface coil(s) and microcoil(s)), so as to overcome the reduced image volume and image quality from the microcoil(s). This situation is enhanced through the use of parallel imaging methods with the combination of surface coil(s) and microcoils(s). Parallel imaging methods acquire MR data in a different way than conventional MRI by measuring sensitivity patterns of coils, filling/building k-space more intelligently, and providing a benefit of correcting non-uniformity of signal patterns. The combination of coils (surface and microcoil) with parallel processing in principal provides a fairly uniform signal intensity pattern, with higher SNR immediately adjacent to the microcoil, where vital information is needed.

A typical microcoil sensitivity pattern is highly non-uniform; it tends to peak immediately adjacent to the microcoil(s) structure and decreases rapidly with distance from the microcoil, typically offering little advantage over surface or head coils at distances of 10 to 20 mm from a typical microcoil. This very non-uniform SNR pattern (from microcoils) requires image correction for visualization of structures of interest. Typical approaches include calculating the correction factors based on previously measured patterns, based on low-pass filtered images, or based on theoretical simulations. Such correction methods are inefficient and cope poorly with variations due to coil orientation effects, coil loading and tuning changes, and coil manufacturing imperfections. Such corrections render background outside the coil sensitivity pattern either black or noisy. Present systems are not provided with capabilities for using surface coils around patients in combination with insertable devices incorporating microcoils to provide their operation in concert with parallel processing. The surface coils should be distributed appropriately on the patient where such distribution might include (e.g., heads) size, shape and pattern of coils on the head around the insertion point and depth to which the microcoil(s)/device(s) are to be placed, e.g., helmet structure including appropriate surface coils for device to be inserted through burr hole into the brain or an elliptical or circular pattern disposed around the head, corresponding to depth of insertion. Signals from each coil can, for example, be designed to predominate over each sub-area or the volume where images are sought. Parallel imaging methods will perform better using the appropriate coil(s) for the particular anatomy or procedure intended. For example, if an MR system with 16 receiver channels is to be used, then there could be 16−n independent surface coils in the helmet and n microcoils in the insertable device.

Viswanathan (U.S. Pat. No. 6,560,475), which is incorporated herein in its entirety by reference for its technical and enabling disclosure on materials and constructions describes microcoil designs that enable unique RF response field profiles that are particularly useful in MRI imaging procedures, particularly where fields of view outside of the medical device are desirable. These devices are particularly for use within an organism, the device comprising an element having at least one RF receiver, the coils of said microcoils defining a cross-section that lies in a plane oriented at 0 to 90 (or 0 to 80) degrees to the longest axis of the device. Another way of describing the device is as a device for use in an organism, the device comprising an element having at least one wound microcoil with at least three windings on the microcoil. Each winding has an aspect ratio of greater than one. The aspect ratio of each winding is measured as the ratio of longest to shortest dimension in a cross section situated approximately transverse to the winding axis of the coil windings, the winding axis also being transverse to the longest axis of said device. Another way of describing the device for use within an organism is as a device comprising an element having at least one RF receiver microcoil, the coils of the microcoils defining a cross-sectional contour having an alignment value of at least 0.75 with the longest axis of the device. The coil windings of the microcoil may have the cross-section comprise a geometric shape, such as a curvilinear shape, a polygon (regular or irregular), or a polygon where corners on the polygon are softened (e.g., slightly rounded). The device may comprise a catheter having at least one lumen. At least one microcoil should be located with its longest dimension defining a longitudinal spatial extent (direction) parallel to the at least one lumen and the coils having a conductor thickness of greater than 0.01 mm and less than 2.4 mm. The described device includes, by way of non-limiting examples, a device for use within an organism, the device comprising an element having at least one RF receiver having at least one wound microcoil with at least one coil having at least three windings in the at least one coil, the coils of the at least one microcoil defining a cross section that lies in a plane oriented at 0-80 degrees to the longest axis of the device, the windings having an aspect ratio of greater than one. Also described are devices for use in an organism, the device having a longest axis, the device comprising an element having at least one wound microcoil with at least three coil windings on the at least one wound microcoil, each coil winding having an aspect ratio of greater than one, the aspect ratio of each coil winding being measured as the ratio of longest to shortest dimension in a cross section situated transverse to the winding axis of the coil windings, the winding axis also being transverse to the longest axis of said device.

U.S. Pat. No. 6,587,706 (Viswanathan), which is incorporated herein in its entirety by reference for its technical and enabling disclosure on materials and constructions, describes microcoils that can be used in medical devices to enhance RF response signals and to create fields to enhance imaging capability in MRI imaging systems. One microcoil design includes a device to be inserted into a patient comprising a solid body having at least one pair of radially opposed microcoils physically associated with the solid body, each microcoil having an outside microcoil diameter of 6 mm or less, individual windings of each microcoil together defining a geometric plane for each microcoil, and the plane of each microcoil being parallel to the plane of another microcoil in the pair of radially opposed microcoils. This reference describes a method of generating an electromagnetic RF receptive field exterior to a device comprising: providing a device comprising a solid body having a geometric center and a distal end, with at least one pair of radially opposed microcoils physically associated with the solid body at said distal end, each microcoil having an outside microcoil diameter of 6 mm or less, the individual windings of said each microcoil defining a geometric plane, and the plane of each microcoil being parallel to the plane of another microcoil in the pair of radially opposed microcoils; providing a radio frequency field around said device; and generating an RF responsive spatial region extending from said at least one pair of opposed microcoils toward or beyond said distal end. Also described are devices adapted to be inserted into a patient, the device comprising a solid body having at least one pair of opposed microcoils physically associated with the solid body, each microcoil having an outside microcoil diameter of 6 mm or less, collective individual windings of said each microcoil defining a geometric plane, and the plane of each microcoil being parallel to the plane of another microcoil in the pair of opposed microcoils; and a device adapted to be inserted into a patient, the device comprising a solid body having at least one pair of opposed microcoils physically associated with the solid body, each microcoil having an outside microcoil diameter of 6 mm or less, at least 50 number % of individual windings of said each microcoil lying within a geometric plane, and the geometric plane of each microcoil being parallel to the plane of another microcoil in the pair of opposed microcoils, and there being at least four windings within each microcoil in said at least one pair of opposed microcoils. There is also described a method of generating an electromagnetic RF responsive field exterior to an device comprising: providing a device comprising a solid body having a geometric center and a distal end, with at least one pair of opposed microcoils physically associated with the solid body at said distal end, each microcoil having an outside microcoil diameter of 6 mm or less, the geometrically averaged position of individual windings of said each microcoil defining a geometric plane, and the plane of each microcoil being parallel to the plane of another microcoil in the pair of opposed microcoils; providing a changing magnetic field around said at least one pair of opposed microcoils to generate an electrical signal in said microcoils; and a responsive field from said microcoils extending from said at least one pair of opposed microcoils towards or beyond said distal end. An alternative perspective of a method of that Viswanathan Patent is a method of generating an electromagnetic RF responsive field exterior to an device comprising: providing a device comprising a solid body having a geometric center and a distal end, with at least one pair of opposed microcoils physically associated with the solid body at said distal end, each microcoil having an outside microcoil diameter of 6 mm or less, at least 50 number % of individual windings of said each microcoil lying within a geometric plane; causing a change in a magnetic field around said device to generate a field response from said microcoils; and a responsive field extending from said at least one pair of opposed microcoils towards or beyond said distal end.

U.S. Pat. No. 6,487,437 (Viswanathan et al.), which is incorporated herein in its entirety by reference for its technical and enabling disclosure on materials and constructions, describes a microcoil configuration, preferably on a medical device to be inserted into a patient, that has an opposed pair of microcoils. At least one or each microcoil of the opposed pair of microcoils has at least a region where a diameter circumscribed by a first winding is greater than the diameter circumscribed by at least one complete second winding, especially an adjacent winding displaced from the first winding along an axis or core of the medical device or an axis of the microcoil. The second winding is nearer to or farther from an intermediate region between the microcoils that define the pair of microcoils. For example, it is common to have a connecting (usually straight or non-wound) lead between the two microcoils, and this lead may be used to define an intermediate region. The microcoil configuration with varying circumference between windings (especially adjacent windings) is generally referred to as a dumb-bell or horn configuration because of its general appearance and the individual microcoils are referred to as a horn microcoil, again because of the visual appearance of the microcoil. The configuration of the microcoils assists in defining the properties of an RF responsive field adjacent to the device.

U.S. Pat. No. 5,964,705 (Truwit et al.), which is incorporated herein in its entirety by reference for its technical and enabling disclosure on materials and constructions, describes the use of devices in procedures, especially medical procedures where the events take place under view of Magnetic Resonance Imaging (MRI) systems is becoming more important. Although some general and specific structures have been discussed in the literature and commercialized, little has been done effectively to design devices for MRI procedures for specific tasks. This invention describes a device for use within an organism, the device comprising an element having at least one pair of opposed RF receiver microcoils having a space between each microcoil of the pair of microcoils, the coils of the microcoils may have diameters of less than 2.4 mm. The device may also comprise an element having at least one pair of opposed RF receiver microcoils having a space between each microcoil of the pair of microcoils, the RF receiver microcoils each comprising at least three individual coils, the at least three individual coils of the microcoils having spacing between adjacent microcoils so that spacing between at least two pairs of individual coils within the microcoils differ by at least 10%. Circuitry may be insulated within the device by providing the wires and circuits within different layers in a coaxial layering of components within the catheter. The device may also comprise device an element having at least one pair of opposed RF receiver microcoils having a space between each microcoil of the pair of microcoils, the RF receiver microcoils each comprising at least three individual windings, the at least three individual windings of the microcoils having spacing between adjacent windings so that spacing between at least two pairs of individual windings within the microcoils differ by at least 10%.

U.S. Pat. No. 6,606,513 (Lardo et al.), describes a system, method, and means for an MRI transseptal needle that can be visible on an MRI, can act as an antenna and receive MRI signals from surrounding subject matter to generate high-resolution images and can enable real-time active needle tracking during MRI guided transseptal puncture procedures. To improve a recognized guidewire visualization problem, two approaches have been taught: passive visualization, and active visualization. With the passive visualization approach, the material of the guidewires is modified so that the catheter appears bright or dark on MR images. Unfortunately, in these techniques data acquisition speed is often limited and the position of the guidewire cannot be visualized very accurately as it depends on the signal-to-noise ratio (SNR) of a second remote detector coil (antenna) which may be sub-optimal. In addition, the modification of the material may result in image artifacts distorting the view of neighboring tissue. In the active visualization techniques, the MRI signal is received by an antenna placed at the end of the guidewire that potentially provides high SNR and spatial resolution in the vicinity of the antenna. These types of probes, although not necessarily this particular probe, have also presented problems for clinical applications, since the antennas are often difficult to insert, providing proper shielding from body fluids and tissues has been difficult, and avoiding injury to patients has at times required suboptimally sized probes to be used.

U.S. Pat. No. 6,633,161 (Vaughn), which is incorporated herein in its entirety by reference for its technical and enabling disclosure on materials and constructions, describes an RF coil suitable for use in imaging systems, which coil has a dielectric filled cavity formed by a surrounding conducting enclosure, the conducting enclosure preferably being patterned to form continuous electrical paths around the cavity, each of which paths may be tuned to a selected resonant frequency. The patterning breaks up any currents induced in the coil and shortens path lengths to permit higher frequency, and thus higher field strength operation. The invention also includes improved mechanisms for tuning the resonant frequency of the paths, for selectively detuning the paths, for applying signal to the coil, for shortening the length of the coil and for controlling the field profile of the coil and the delivery of field to the object to the image.

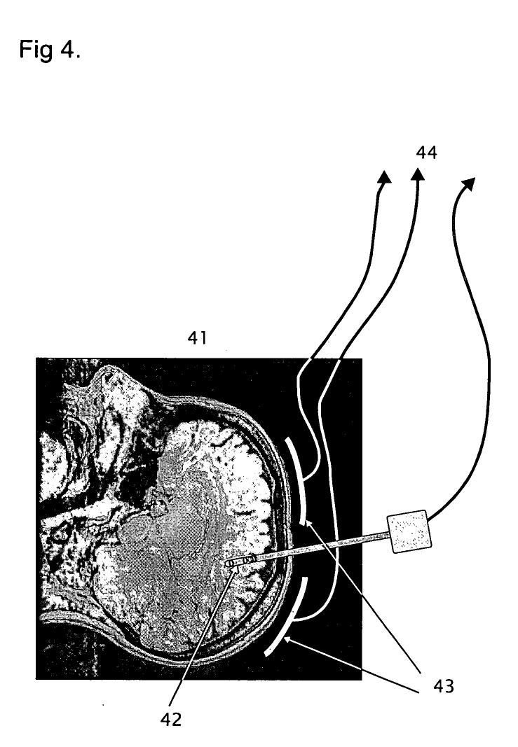

U.S. Pat. Nos. 6,516,211 and 6,128,522 (Acker), which are incorporated herein in their entirety by reference for its technical and enabling disclosure on materials and constructions, describe a magnetic resonance information acquired by a movable magnetic resonance instrument used to monitor hyperthermia treatments such as tissue ablation. The instrument may include both the magnetic resonance equipment and an energy applicator such as a high intensity focused ultrasound unit. The treatment can be conducted under automatic control after the operator marks a treatment volume on an image of the subject, such as a magnetic resonance image acquired using the movable magnetic resonance instrument. The automatic treatment can be based on interpolation of tissue response curves at plural test points near the treatment. One of the coil designs shown in the figures (FIG. 4) and specification uses multiple parallel coils. These inventions do not teach separate receiver channels for each microcoil, or anything about image processing for more than one microcoil.

As illustrated by the accompanying figures, the technology described herein may be practiced in a number of different formats and procedures with conventional or commercial MR equipment in combination with the novel placement distribution and combination of microcoils and external coils. The various types of microcoils and body coils described in the art, patents, trade literature, journals and other published literature may be used within the practices of the present technology.

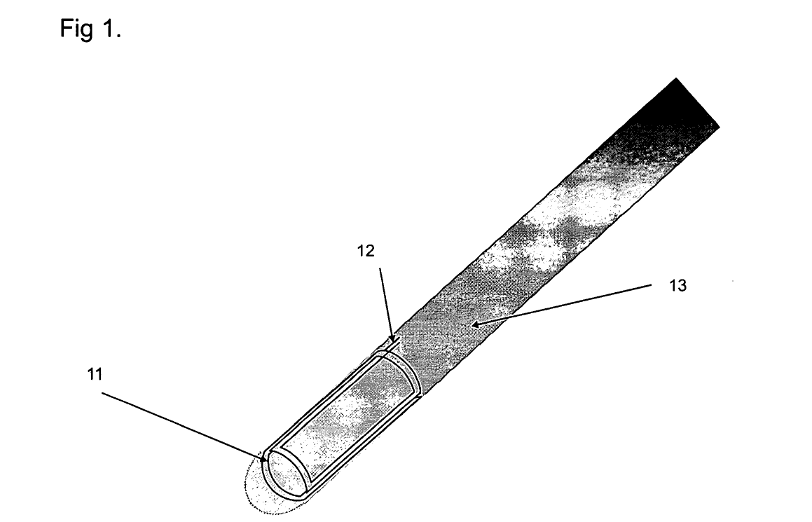

FIG. 1 shows a diagram of part of a catheter (13) incorporating a microcoil (11) with windings near the tip of the catheter shaft. The structure of the coil in this and subsequent figures and discussions may be selected from among known and future developed coil structures and materials, such as, but not limited to the designs and materials specifically described herein. The location of the coils may also be varied to allow for diagnosis- or treatment-specific location of the responsive signals with respect to any medical device on which the coil is located. The leads from the microcoil (12) pass through the catheter to an MR imager. Any commercial microcoil system may be used in combination with the practices of the technology described herein. A number of materials known in the art may be used to manufacture the infusion catheter disclosed by the present invention.

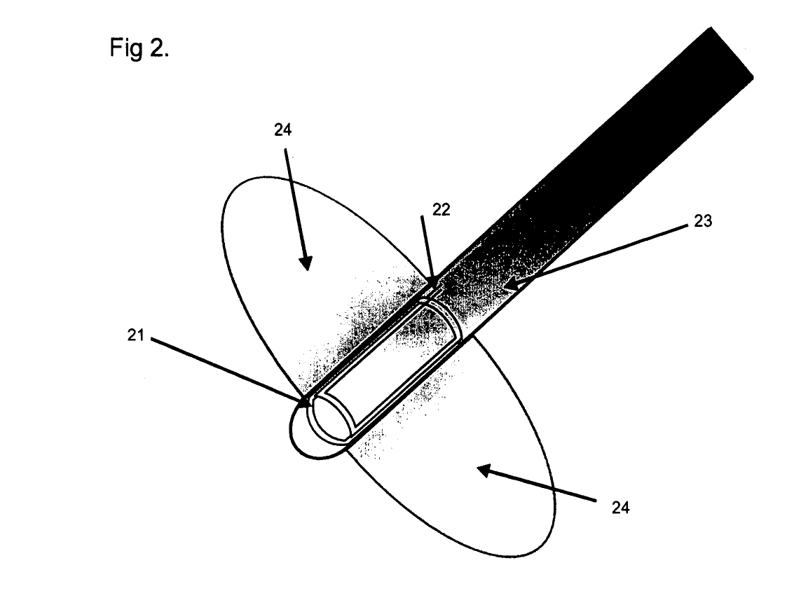

In an embodiment of the apparatus of the invention shown in FIG. 2, a catheter (23) incorporating a microcoil (21) with windings near the tip of the catheter shaft has an RF responsive field (24) radially disposed around the microcoil position. The leads from the microcoil (22) pass along the catheter to an MR imager.

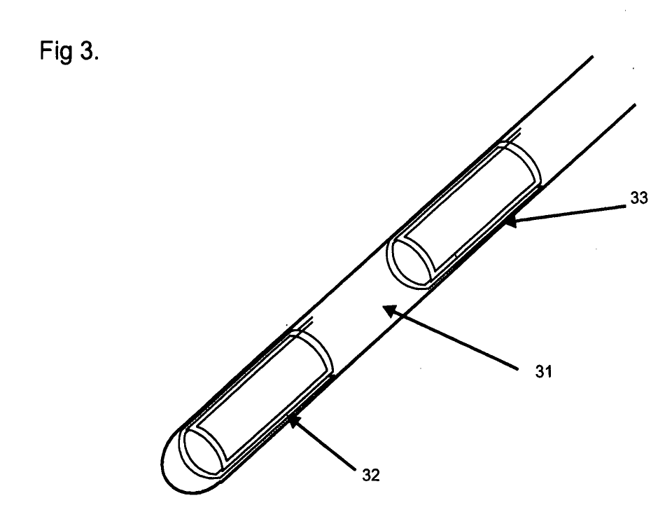

FIG. 3 illustrates another embodiment of the apparatus of the present invention, wherein part of a catheter (31) incorporates two microcoils (32 and 33), one positioned near the distal tip of the catheter and a second microcoil positioned further away from the catheter tip.

FIG. 4 shows an embodiment of the method of the invention for performing an intracranial drug, cell or gene therapy delivery procedure in a human patient (41). A microcoil is shown within an inserted catheter device (42), surrounded by two surface coils (43). The signals (44) from the coils go to separate receiver channels of an MR imager.

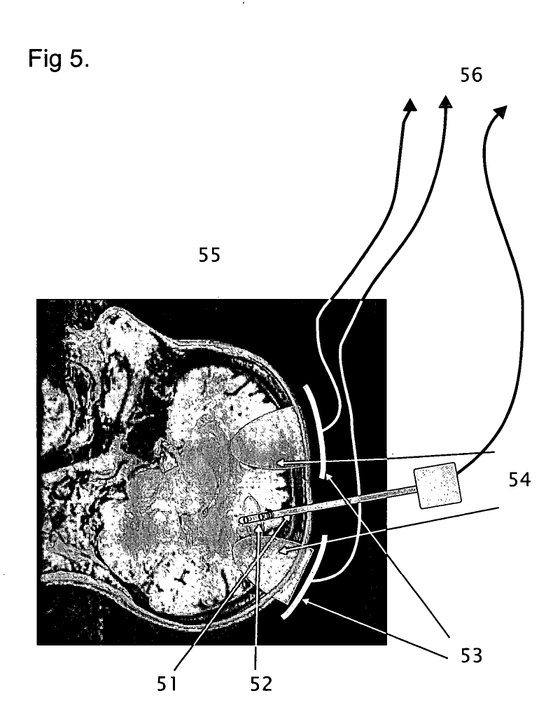

FIG. 5 outlines the head of a patient (55). A microcoil/catheter (51) is shown inserted in the head with an RF responsive field (52). In a further embodiment of the method of the invention, two surface coils (53) are positioned near the insertion point with their RF responsive fields (54). Signals from the surface coils and microcoil (56) go to separate receiver channels of an MR imager.

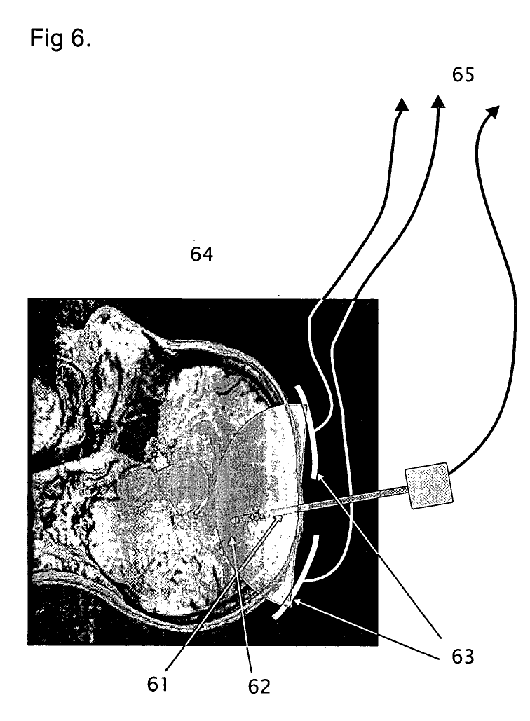

FIG. 6 shows a preferred embodiment of the invention comprising an infusion catheter with microcoil (61) surgically inserted into a human brain (64) in an appropriate location for treatment predetermined by imaging before the invasive procedure. Two surface coils (63) are positioned around the insertion point of the catheter. Signals from the surface coils and microcoil (65) go to separate receiver channels of an MR imager wherein a combined volume (62) is imaged effectively by parallel imaging methods. According to the practices of the technology described herein, the image data from these two distinct sources of information would be combined to provide greater detail in the specific region of concern. This combination of surface coil and microcoil imaging volume would be effected by any imaging or mathematical procedure that would produce a visually useful combination of the data, and would be likely to enhance the resolution in the specific region of concern. These methodologies are well understood by the imaging specialists and radiologists.

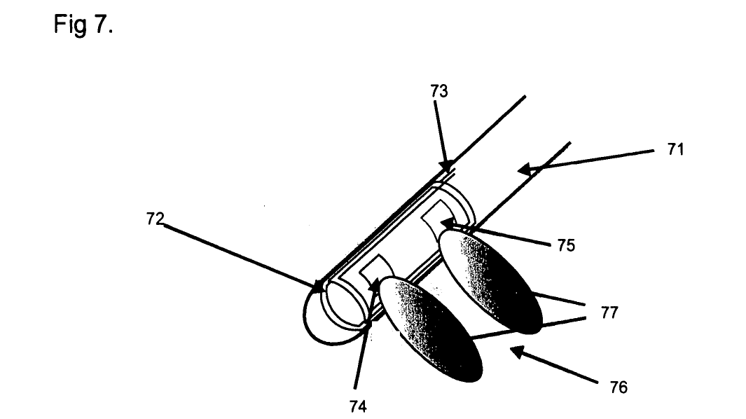

FIG. 7 shows the tip of a catheter (71) containing a microcoil (72) with signal leads (73) extending to an MR imager. Two infusion ports (74) and (75) of an infusion catheter are provided near the catheter tip from which small infusions of therapeutic magnets (77) are dispersed under pressure into the surrounding tissues. According to the invention, a limited field-of-view (76) radially disposed around the microcoil position covers the volume of infusion only during the immediate post-infusion period

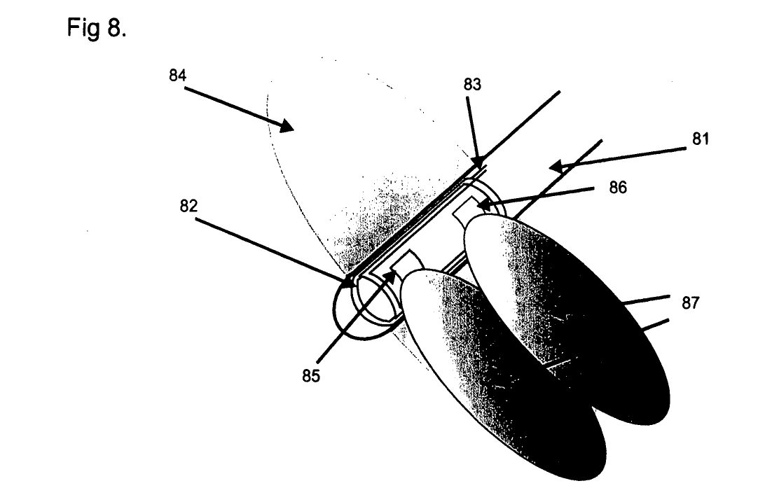

FIG. 8 illustrates an infusion catheter (81) with microcoil (82) with signal leads (83) extending to an MR imager. Two infusion ports (85) and (86) are provided near the catheter tip from which higher volume infusions of therapeutic agents (87) are dispersed under pressure into the surrounding tissues. The RF responsive field (84) radially disposed around the microcoil does not have a sufficiently large field-of-view to cover the entire infusion volume of a diagnostic or therapeutic agent when the infusion volumes are large or when the migration of drugs, cells or gene therapy agents delivered into the brain extends beyond the field-of-view of the microcoil.

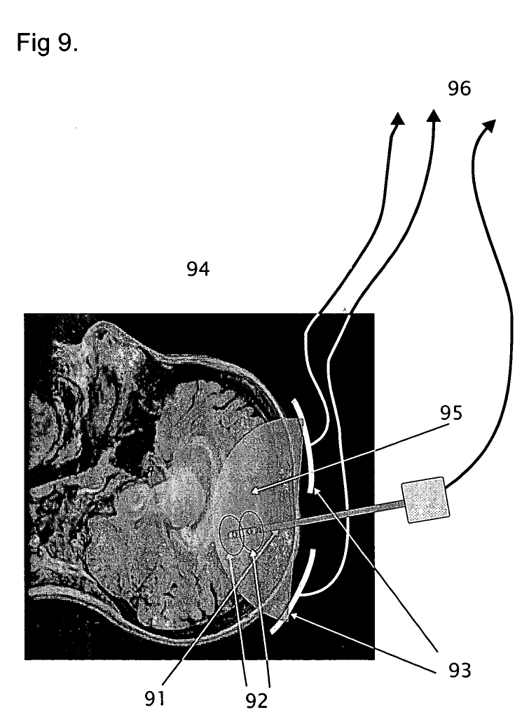

FIG. 9 illustrates a further preferred embodiment of the invention wherein MR responsive signals from a catheter-based microcoil are integrated with MR responsive signals from surface coils positioned on the head using parallel imaging methods. A catheter with microcoil (91) is shown inserted into the brain of a human patient (94). Two surface coils (93) are positioned around the insertion point of the catheter. Signals from the surface coils (93) and microcoil (91) interface with separate receiver channels of an MR imager wherein a combined volume (95) is imaged effectively by parallel imaging methods. The combined volume serves to image larger volume infusions of diagnostic or therapeutic agents (92) from the tip of the catheter which would otherwise exceed the field-of-view of the microcoil. According to the invention, the extended field-of-view of the combination of microcoil and surface coils enables tracking of device location and subsequently enables dynamic MR visualization of the migration of diagnostic or therapeutic agents delivered from the infusion catheter over hours and days after delivery.

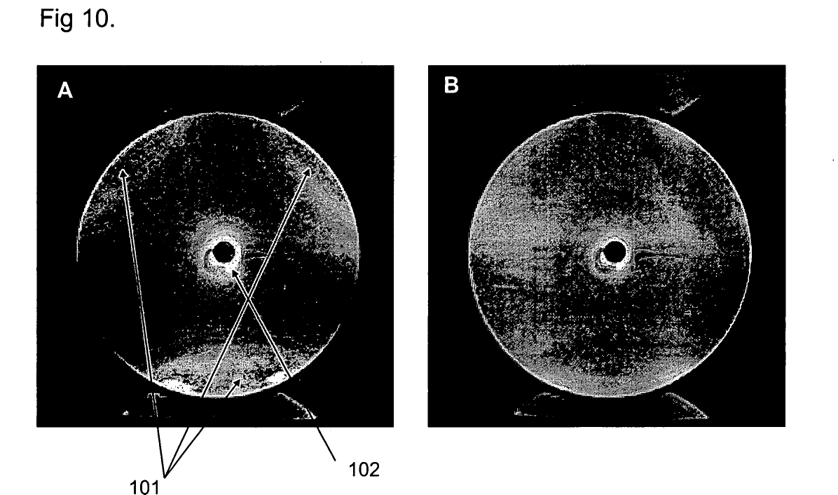

FIG. 10 shows one possible implementation of the invention. (A) is an MR image of a 10-cm-diameter spherical object filled with a tissue-equivalent material. Three surface coils (101) are disposed externally symmetrically about the object and a device with a microcoil (102) is inserted internally into the center of the object, transverse to the plane of the image. (A) is an embodiment wherein the images from the three surface coils and the microcoil are represented individually illustrating their respective RF responsive fields as bright. (B) is an embodiment wherein parallel imaging methods are used to produce an advantageous combined volume from the three surface coils and the internal microcoil, encompassing the entire object in this example.

As illustrated by the descriptions and examples in the preceding figures, the present invention may be practiced by the operation of certain exemplary device embodiments. For example, the technology may be practiced as a method for providing a field of view within a volume of a patient, the method comprising providing at least two RF surface coils that at least in part MR responsively cover the volume, providing at least one MR responsive microcoil within the volume, and simultaneously generating MR responsive fields from the at least two RF surface coils and the at least one microcoil. The data of the RF responsive fields from the surface coils and the at least one microcoil are then integrated with parallel imaging methods. It is desirable that there are at least two or even at least six or even at least eight microcoils present within the volume. As noted earlier, there are numerous parallel processing methods available to the artisan, such as where SENSE or SMASH parallel processing is used to integrate data. The method can be desirably practiced by simultaneously generating MR responsive fields from the at least two RF surface coils and the at least one microcoil in a magnetic field of at least 1.5 Tesla.

The presently described technology may also be viewed or described from the perspective of comprising a method for enhancing the signal to noise ratio of a field of view within a volume of a patient by providing at least two RF surface coils that at least in part MR responsively cover the volume, providing at least one MR responsive microcoil within the volume, simultaneously generating MR responsive fields from the at least two RF surface coils and the at least one microcoil, then integrating data of the RF responsive fields with parallel imaging methods.

The presently described technology may also be viewed or described from the perspective of comprising a system for the provision of visual images of a volume of a patient comprising magnetic resonance imaging apparatus, at least two surface coils for positioning about a patient, and a medical instrument having at least one microcoil thereon, the magnetic resonance imaging apparatus providing data to a processor that will integrate field magnetic responsive data from the at least two surface coils and the microcoil to provide the visual images of a patient.

The presently described technology may also be viewed or described from the perspective of comprising a medical device having a longitudinal axis, the device having at least one RF microcoil system and at least one surface coil system, the position of the at least one RF microcoil system and the at least one surface coil system with respect to the longitudinal axis defining regions where different field volumes are provided by the at least one RF microcoil system and the at least one surface coil system. The medical device may have the surface and microcoil systems each connected with at least one preamplifier to communicate between the at least one surface and one microcoil system and a signal receiving system or there are at least two surface coil systems or there are at least two distinct RF microcoil systems.

The presently described technology may also be viewed or described from the perspective of comprising a method of providing a magnetic resonance image comprising: a) providing a medical device having a longitudinal axis, the device having at least one RF microcoil and at least one surface coil, the position of the at least one microcoil and the at least one surface coil with respect to the longitudinal axis defining regions where different field volumes are provided by the at least one microcoil and at least one surface coil; b) placing the device within a magnetic resonance imaging field; c) generating two distinct imaging fields, one at least one field each from the at least one microcoil and at least one surface coil; and d) integrating images of the two distinct fields into a single image. In practicing this method, each surface and microcoil system may have at least one preamplifier in communication between the at least one surface and at least one microcoil system and a signal receiving system.

Although many individual examples of components for the commercially available surface coils, microcoils, MR devices, and combinations of these components have been provided in this disclosure, the examples are not intended to be limiting in the disclosure of the generic systems, methods and apparatus used in the practice of the described and claimed technology.

What is claimed:

1. A method and apparatus for providing an MR imaging field of view within a target MR imaging volume of a patient comprising providing at least two RF surface coils that at least in part MR responsively cover the volume, providing at least one MR responsive microcoil within the volume, simultaneously generating MR responsive fields from the at least two RF surface coils and the at least one microcoil, then integrating data of the RF responsive fields with parallel imaging and processing methods.

2. The method of claim 1 wherein at least two microcoils MR responsively cover said target imaging volume.

3. The method of claim 1 wherein at least 4 surface coils MR responsively cover said target imaging volume.

4. The method of claim 2 wherein at least 4 surface coils MR responsively cover said target imaging volume

5. The method of claim 1 wherein at least 8 surface coils MR responsively cover said target imaging volume.

6. The method of claim 2 wherein at least 6 microcoils MR responsively cover said target imaging volume.

7. The method of claim 2 wherein parallel MR imaging and processing methods are used to integrate said imaging data.

8. The method of claim 3 wherein parallel MR imaging and processing methods are used to integrate said imaging data.

9. The method of claim 4 wherein parallel MR imaging and processing methods are used to integrate said imaging data.

10. The method of claim 5 wherein parallel MR imaging and processing methods are used to integrate said imaging data.

11. The method of claim 6 wherein parallel MR imaging and processing methods are used to integrate said imaging data.

12. The method of claim 3 wherein parallel MR imaging and processing methods are used to integrate said imaging data.

13. The method of claim 4 wherein parallel MR imaging and processing methods are used to integrate said imaging data.

14. The method of claim 1 wherein simultaneously generating MR responsive fields from the at least two RF surface coils and the at least one microcoil is carried out in a magnetic field of at least 0.5 Tesla.

15. The method of claim 2 wherein simultaneously generating MR responsive fields from the at least two RF surface coils and the at least one microcoil is carried out in a magnetic field of at least 0.5 Tesla.

16. The method of claim 3 wherein simultaneously generating MR responsive fields from the at least two RF surface coils and the at least one microcoil is carried out in a magnetic field of at least 0.5 Tesla.

17. The method of claim 1 wherein simultaneously generating MR responsive fields from the at least two RF surface coils and the at least one microcoil is carried out in a magnetic field of at least 1.5 Tesla.

18. A method for enhancing the signal to noise ratio of a field of view within a volume of a patient comprising providing at least two RF surface coils that at least in part MR responsively cover the target imaging volume, providing at least one MR responsive microcoil within the volume, simultaneously generating MR responsive fields from the at least two RF surface coils and the at least one microcoil, then integrating data of the RF responsive fields with parallel imaging and processing methods.

19. A system for the provision of visual images of a target volume within a patient comprising magnetic resonance imaging apparatus, at least two surface coils for positioning about a patient, and a medical instrument having at least one microcoil thereon, the magnetic resonance imaging apparatus providing data to a processor that will integrate field magnetic responsive data from the at least two surface coils and the microcoil to provide the visual images of a patient.

20. A medical device having a longitudinal axis, the device having at least one RF microcoil system and at least one surface coil system, the position of the at least one RF microcoil system and the at least one surface coil system with respect to the longitudinal axis defining regions where different field volumes are provided by the at least one RF microcoil system and the at least one surface coil system.

21. The medical device of claim 20 wherein the surface microcoil system is connected with at least one preamplifier in communication between the at least one surface microcoil system and a signal receiving system

22. The medical device of claim 20 wherein there are at least two surface coil systems.

23. The medical device of claim 20 wherein there are at least two distinct RF microcoil systems.

24. The medical device of claim 23 wherein there are at least two surface coil systems.

25. A method of providing a magnetic resonance image comprising:
a) providing a medical device having a longitudinal axis, the device having at least one RF microcoil and at least one surface coil, the position of the at least one microcoil and the at least one surface coil with respect to the longitudinal axis defining regions where different field volumes are provided by the at least one microcoil and at least one surface coil;
b) placing the device within a magnetic resonance imaging field;
c) generating two distinct imaging fields, one at least one field each from the at least one microcoil and at least one surface coil; and
d) integrating images of the two distinct fields into a single image.

26. The method of claim 25 wherein the distinct imaging fields overlap.

27. The method of claim 25 wherein the distinct fields do not overlap.

28. The method of claim 25 wherein the surface microcoil system has at least one preamplifier in communication between the at least one surface microcoil system and a signal receiving system.

29. A method and apparatus for providing an extended MR imaging field of view within a volume of a human body undergoing direct drug therapy, cell therapy, or gene therapy, comprising providing at least two RF surface coils that at least in part M responsively cover the volume, providing at least one MR responsive microcoil within the volume, simultaneously generating MR responsive fields from the at least two RF surface coils and the at least one microcoil, then integrating data of the RF responsive fields with parallel imaging and processing methods.
